(19) United States
(12) Patent Application Publication
(10) Pub. No.: US 2009/0306032 A1
(43) Pub. Date: Dec. 10, 2009

(54) Use of solid lipid nanoparticles Comprising Cholesteryl Propionate and/or Cholesteryl Butyrate

(76) Inventor: Maria Rosa Gasco, Torino (IT)

Correspondence Address: ABELMAN, FRAYNE & SCHWAB, 666 THIRD AVENUE, 10TH FLOOR, NEW YORK, NY, 10017, US

(21) Appl. No.: 11/921,634

(22) PCT Filed: May 31, 2006
(86) PCT No.: PCT/EP2006/062783

§ 371 Date: Jul. 23, 2009

(30) Foreign Application Priority Data
Jun. 1, 2005 (IT) MI2005A001024

(51) Int. Cl. A61K 31/575; A61P 29/00

(52) U.S. Cl. 514/182; 977/773

(57) ABSTRACT

The present invention refers to the use of solid lipid nanoparticles (SLN) obtained from warm microemulsions, containing cholesteryl propionate and/or cholesteryl butyrate, for preparation of a medicament for the prevention and treatment of vascular or inflammatory pathologies.

### STATE OF THE ART

Short chain fatty acids are small natural molecules endowed with strong modulatory activities on cell growth and differentiation.

In particular, there are several experimental evidences that sodium propionate and butyrate are therapeutic agents of remarkable interest for prevention and therapy of vascular and inflammatory pathologies.

For instance, physiological concentrations of sodium butyrate were shown to inhibit proliferation of vascular smooth muscle cells without inducing cytotoxicity (Feng P. et al Cell Prolif. 29; pag. 231-241 (1996); Raganna et al. Arteriosclerosis Thrombosis and Vascular Biology 15, pag. 2273-2283 (1995)). Excessive proliferation of vascular smooth muscle cells (VSMC) represents a critical element in development of several vascular pathologies, especially atherosclerosis and postangioplastic restenosis.

Moreover, there are experimental evidences of the activity of butyric acid also in inflammatory pathologies.

For instance, some authors have shown that butyric acid improves inflammation in ulcerative colitis (Luhrs H. et al, Scandinavian Journal of Gastroenterology 37; pag. 458-466 (2002)) and pre-clinical and clinical studies suggest that sodium butyrate is effective in the treatment of irritable bowel syndrome (Venkatraman H. et al. Febs Letters 554, pag. 88-94 (2003)).

Patent application EP1133286 describes solid lipid nanoparticles containing cholesteryl butyrate acting as lipid pro-drug of butyric acid and the use of said particles in the treatment of tumor pathologies and mediterranean anemia.

### SUMMARY OF THE INVENTION

Now the applicant has surprisingly found that solid lipid nanoparticles containing cholesteryl propionate and/or cholesteryl butyrate possess antiproliferative and anti-inflammatory activities that are surprisingly higher than the corresponding short chain fatty acids.

Therefore, the present invention relates to the use of said solid nanoparticles for prevention and treatment of vascular and/or inflammatory pathologies.

### DETAILED DESCRIPTION OF THE INVENTION

Object of the present invention is the use of solid lipid nanoparticles (SLN) obtained from warm microemulsions, and containing cholesteryl propionate and/or cholesteryl butyrate, in the preparation of a medicament for prevention and treatment of vascular and/or inflammatory pathologies.

According to a preferred embodiment of the present invention, said pathologies are chosen from the group comprising atherosclerosis, restenosis, and inflammatory bowel diseases, as for instance Crohn's disease and ulcerative colitis.

Preferably, the nanoparticles of the present invention contain cholesteryl butyrate.

According to a preferred embodiment, the particles used in the present invention are the same as those described in EP1133286.

Said nanoparticles have an average diameter smaller than 400 nm, preferably comprised between 100 and 200 nm, and a polydispersion index comprised between 0.10 and 0.50, and they can be obtained from warm microemulsions of cholesteryl propionate and/or cholesteryl butyrate by the process already described in EP1133286.

According to a particularly preferred embodiment of the present invention, solid lipid nanoparticles contain between 15% and 46% by weight, of cholesteryl propionate and/or cholesteryl butyrate in association with pharmaceutically acceptable surfactants or co-surfactants agents.

Moreover, the present invention relates to a therapeutic method for treatment of vascular and inflammatory pathologies, comprising the administration of pharmacologically effective amounts of said nanoparticles. Said administration is preferably made through the oral or rectal route.

As shown in detail in the following examples, solid lipid nanoparticles containing cholesteryl butyrate exhibit an antiproliferative activity on vascular smooth muscle cells (VSMC) and an inhibiting activity on activation of polymorphonuclear cells that is surprisingly higher than the activity observed with butyric acid.

Adesion of polymorphonuclear cells (PMN) to vascular cells is a step in tissue leukocyte infiltration during inflammation.

Therefore, the nanoparticles of the invention show a higher effectiveness than butyric acid in the treatment of vascular and inflammatory pathologies.

### EXAMPLE 1

### Preparation of Cholesteryl Butyrate SLN

A microemulsion consisting of 15% of Epikuron 200® mixture (soybean lecithin containing as minimum 92% phosphatidylcholine), 12% cholesteryl butyrate, 3% taurocholate, 11% butanol and 59% water was prepared.

In detail, Epikuron 200® and cholesteryl butyrate were heated until fusion, at about 85° C. A warm aqueous solution of taurocholate and butanol was then added under agitation, thus obtaining a clear system.

The so obtained microemulsion was dispersed in cold water at 2-3° C. and the dispersion was washed by diafiltration using a membrane with a cut-off of 100,000. The dispersion was then hot sterilized (15 minutes at 121° C.).

The average diameter of the nanoparticles was determined, that was of 150 nm, with a polydispersion index of 0.215.

### EXAMPLE 2

### Adhesion Assay

Human umbilical vein endothelial cells (HUVEC cells) were suitably treated, grown to confluence in 24 well plates, washed and maintained for one day in M199 medium supplemented with 10% BCS (Bovine Calf Serum).

Polymorphonuclear cells (PMN) were prepared from citrated venous blood obtained from healthy volunteers. Polymorphonuclear cells (PMN), at the concentration of 10⁷ cells/ml were labeled with fluorescein diacetate (5 μg/ml) for 30 minutes at 37° C., washed with BSS and plated at the concentration of 10⁶ cells per well in a final volume of 0.25 ml of BSS.

The antiadhesive effects of cholesteryl butyrate nanoparticles prepared in example 1 and of sodium butyrate were measured by an adhesion assay. In detail, human polymorphonuclear cells and HUVEC endothelial cells were incubated with increasing concentrations of cholesteryl butyrate or sodium butyrate nanoparticles in presence of a substance, PAF or IL-1β, which induces adhesion of PMN cells to endothelial HUVEC cells. Adhesion was then assessed by fluorescence microimaging.

a) The effect of cholesteryl butyrate nanoparticles prepared as in example 1, or of sodium butyrate, on adhesion of polymorphonuclear cells to HUVEC cells was examined in presence of the adhesion stimulus provided by PAF (10⁻⁷M). Polymorphonuclear cells and HUVEC cells were co-incubated in presence of PAF and of increasing concentrations, ranging between 10⁻⁸ and 10⁻⁵, of cholesteryl butyrate (in the form of nanoparticles of Example 1) or of sodium butyrate, for 5 minutes at 37° C.

A nearly maximal adhesion of PMN to HUVEC cells is obtained in presence of 10⁻⁷M PAF concentration; an almost complete inhibition (81%) of the adhesion and a IC₅₀ of 8.0×10⁻⁸ is obtained in presence of cholesteryl butyrate at a concentration of 10⁻⁵M, while 74% inhibition and an IC₅₀ of 4×10⁻⁷ M is obtained for the same concentration (10⁻⁵) of sodium butyrate.

b) The effect of cholesteryl butyrate nanoparticles prepared in example 1 or of sodium butyrate on adhesion of polymorphonuclear cells to HUVEC cells was also examined in presence of the adhesion stimulus provided by IL-1β (0.05 ng/ml). Polymorphonuclear cells and HUVEC cells were co-incubated for 4 hours at 37° C. in presence of IL-1β and of increasing concentrations, ranging from 10⁻⁸ to 10⁻⁵, of cholesteryl butyrate (in the form of nanoparticles of Example 1) or of sodium butyrate.

Eighty percent inhibition of the adhesion is obtained in presence of 10⁻⁵ M cholesteryl butyrate concentration, while 22% inhibition of the adhesion is obtained with butyrate at the same concentration.

### EXAMPLE 3

### Growth Inhibition of Vascular Smooth Muscle Cells (VSMC Cells)

Rat VSMC cells were grown in Medium 198 supplemented with 10% Fetal Bovine Serum (FBS), 4 mM glutamine, 100 units/ml sodium penicillin, 100 μg/ml streptomycin sulphate and 0.25 μg/ml amphotericin B.

Said cells were then transferred to 24 well plates at a density of 1×10⁴ cells per well and left to adhere overnight. The next day, cholesteryl butyrate nanoparticles of Example 1 were added to the cells, using a cholesteryl butyrate concentration of 10⁻⁵ M. The medium was changed every 48 hours. Cells subjected to the same treatment in the absence of cholesteryl butyrate nanoparticles were used as control. The number of cells in both samples was determined after 6 days of treatment.

There was a clear inhibition of smooth muscle cells by cholesteryl butyrate. In fact, while a 15-fold increase of VSMC cells was observed in the control, such increase turned out to be 4-fold in the presence of the nanoparticles of the invention.

1.-6. (canceled)

7. A medicament comprising solid lipid nanoparticles (SLN) containing a therapeutically effective amount of a compound selected from the group consisting of cholesteryl propionate cholesteryl butyrate, and mixtures thereof, in combination with members selected from the groups consisting of suitable excipients, diluents, and mixtures thereof, wherein said solid nanoparticles are obtained from warm suspensions.

8. The medicament according to claim 7 for the treatment of vascular and/or inflammatory phatologies.

9. The medicament according to claim 7 wherein said nanoparticles contain cholesteryl butyrate.

10. The medicament according to claim 8 wherein said phatologies are selected from the group consisting of atherosclerosis, restenosis and inflammatory bowel diseases.

11. The medicament according to claim 10 wherein said inflammatory bowel diseases are Crohn's disease and ulcerative colitis.

12. The medicament according to claim 7 wherein said nanoparticles have an average diameter lower than 400 nm and a polydispersion index comprised between 0.10 and 0.50.

13. The medicament according to claim 7 wherein said nanoparticles contain, on a weight basis, between 15% and 46% cholesteryl propionate, cholesteryl butyrate, and mixtures thereof, associated with pharmaceutically acceptable surfactants and co-surfactants.
